(19) United States
(12) Patent Application Publication
(10) Pub. No.: US 2026/0224121 A1
(43) Pub. Date: Aug. 6, 2026

(54) SYSTEM AND METHOD FOR ESTIMATION OF METABOLIC COSTS FROM CARDIAC MEASUREMENTS

(71) Applicant: GEORGIA TECH RESEARCH CORPORATION, Atlanta, GA (US)

(72) Inventors: Woon-Hong YEO, Atlanta, GA (US); Myunghee KIM, Chicago, IL (US)

(21) Appl. No.: 19/150,388

(22) PCT Filed: Jan. 25, 2024
(86) PCT No.: PCT/US2024/012978

§ 371 Date: Jul. 23, 2025

Related U.S. Application Data
(60) Provisional application No. 63/481,540, filed on Jan. 25, 2023.

(51) Int. Cl. A61B 5/024; A61B 5/00; A61B 5/0245; A61B 5/256; A61B 5/257; A61B 5/282

(52) U.S. Cl. CPC A61B 5/02405; A61B 5/0015; A61B 5/0245; A61B 5/256; A61B 5/257; A61B 5/282; A61B 5/4866; A61B 5/681; A61B 5/7264; A61B 5/7475

(57) ABSTRACT

An exemplary system and method are provided that can determine a measure of metabolic cost from heart rate variability data acquired from cardiac signals using a trained machine learning model. The exemplary system and method can employ cardiac signals acquired via ECG equipment or from wearable sensor device.

### CROSS-REFERENCE TO RELATED APPLICATIONS

This application claims the benefit of priority to U.S. Provisional Application No. 63/481,540, filed Jan. 25, 2023, which is incorporated by reference herein in its entirety.

### GOVERNMENT SUPPORT CLAUSE

This invention was made with government support under Grant No. 2024742 awarded by the National Science Foundation. The Government has certain rights in the invention.

### BACKGROUND

The US Bureau of Labor Statistics (USBLS) in 2011 reported more than 310,000 cases of musculoskeletal disorders (MSD) in the work environment; in 2018, the number of reported cases decreased to 277,000, a decrease of about 1.5% per year. The USBLS highlights laborers and freight, stock, and material movers are the most vulnerable class of workers to have musculoskeletal injuries where overexertion from repetitive action involving squatting and lifting creates fatigue in workers, causing MSDs.

Activities and physical effort are commonly estimated using a metabolic rate through indirect calorimetry to capture breath information. Example systems for indirect calorimetry can employ a respiratory mask to collect oxygen intake and carbon dioxide outtake in a controlled environment. Although respirometry is the gold standard for estimating metabolic costs, the method requires a heavy, bulky, and rigid system, e.g., in very specialized facilities.

There is a benefit to improving biophysical signal acquisition, particularly for the determination of metabolic cost.

### SUMMARY

An exemplary system and method are provided that can determine a measure of metabolic cost, e.g., from heart rate variability data acquired from cardiac signals, using a trained machine learning model. The exemplary system and method can employ cardiac signals acquired via ECG equipment or from a wearable sensor device.

In some embodiments, the wearable sensor device includes a soft, flexible bioelectronic system. A study was conducted that developed and evaluated the exemplary method and system in the context of an exosuit evaluation for a set of activities, e.g., walking, running, and squatting. The exemplary system and method can be used to provide a measure of metabolic cost for activities for activity or labor research as well as to monitor for labor-associated injury.

The exemplary system and method can be used in field-deployable platforms that can measure wireless real-time physiological signals.

In an aspect, a system is disclosed comprising: a processor; and a memory having instructions stored thereon, wherein the instructions when executed by the processor causes the processor to: receive a cardiac signal data set acquired by a electrode measurement device placed on a subject while the subject was performing an activity; determine heart rate variability signal or value using the cardiac signal data set; and determine, via a trained machine learning model, a value for metabolic cost for the activity, wherein the trained machine learning model was trained using metabolic cost data and heart rate variability data, wherein the determined value for metabolic cost is made accessible to be used to an evaluation of the activity.

In some embodiments, the cardiac signal data set is acquired from ECG equipment or a wearable sensor device.

In some embodiments, the wearable sensor device includes an array of stretchable electrodes, the array comprising a first side and a second side, a plurality of interconnectors joining the electrodes of the array of stretchable electrodes, and an adhesive patch on the first side of the array of stretchable electrodes, the adhesive patch configured to hold the second side of the array of stretchable electrodes to a surface of a patient's skin.

In some embodiments, the instructions, when executed by the processor, cause the processor to determine via a second trained machine learning model a motion classification output from an acquired motion signal data set acquired via the electrode measurement device.

In some embodiments, the instructions, when executed by the processor, cause the processor to output the value of the metabolic cost in a graphical user interface or a report to be used for an evaluation of the activity.

In some embodiments, the system is configured as a smartwatch or smartphone.

In some embodiments, the system is implemented in cloud infrastructure.

In another aspect, a method is disclosed comprising: receiving a cardiac signal data set acquired by an electrode measurement device placed on a subject while the subject was performing an activity; determining heart rate variability signal or value using the cardiac signal data set; and determining, via a trained machine learning model, a value for metabolic cost for the activity, wherein the trained machine learning model was trained using metabolic cost data and heart rate variability data, wherein the determined value for metabolic cost is made accessible to be used to an evaluation of the activity.

In some embodiments, the cardiac signal data set is acquired from ECG equipment or a wearable sensor device.

In some embodiments, the wearable sensor device includes an array of stretchable electrodes, the array comprising a first side and a second side, a plurality of interconnectors joining the electrodes of the array of stretchable electrodes, and an adhesive patch on the first side of the array of stretchable electrodes, the adhesive patch configured to hold the second side of the array of stretchable electrodes to a surface of a patient's skin.

In some embodiments, the method further includes determining via a second trained machine learning model a motion classification output from an acquired motion signal data set acquired via the electrode measurement device.

In some embodiments, the method further includes outputting the value of the metabolic cost in a graphical user interface or a report to be used for an evaluation of the activity.

In some embodiments, the system is configured as a smartwatch or smartphone.

In some embodiments, the system is implemented in cloud infrastructure.

In some embodiments, the method includes acquiring the cardiac sensor at the electrode measurement device placed on the subject while the subject was performing the activity; and transmitting the acquired cardiac sensor to an analysis system on a remote computing device to perform the determining the value for metabolic cost for the activity.

In some embodiments, the method includes acquiring the cardiac sensor at the electrode measurement device placed on the subject while the subject was performing the activity; and transmitting the acquired cardiac sensor to a computing device that then transmits the acquired cardiac sensor to an analysis system on a remote computing device to perform the determining the value for metabolic cost for the activity, wherein the determined value for the metabolic cost is transmitted from the remote computing device to the computing device.

In some embodiments, the value of the metabolic cost is employed as a measure of physical exertion.

In another aspect, a non-transitory computer readable medium is disclosed having instructions stored thereon, wherein the instructions when executed by a processor causes the processor to: receive a cardiac signal data set acquired by a electrode measurement device placed on a subject while the subject was performing an activity; determine heart rate variability signal or value using the cardiac signal data set; and determine, via a trained machine learning model, a value for metabolic cost for the activity, wherein the trained machine learning model was trained using metabolic cost data and heart rate variability data, wherein the determined value for metabolic cost is made accessible to be used to an evaluation of the activity.

In some embodiments, the cardiac signal data set is acquired from ECG equipment or a wearable sensor device.

In some embodiments, the instructions, when executed by the processor, cause the processor to output the value of the metabolic cost in a graphical user interface or a report to be used for an evaluation of the activity.

### BRIEF DESCRIPTION OF DRAWINGS

The components in the drawings are not necessarily to scale relative to each other. Like reference, numerals designate corresponding parts throughout the several views.

FIGS. 1A and 1B each shows an example device configured to determine metabolic cost estimation from cardiac signals in accordance with an illustrative embodiment.

FIGS. 2A and 2B each shows example methods to calculate metabolic cost estimations from cardiac signals in accordance with an illustrative embodiment.

FIGS. 3A-3D show an example soft flexible bioelectronic system as a wearable sensor device employed in a study that used an ankle-foot-orthosis (AFO) exoskeleton to estimate metabolic costs and physical effort.

FIGS. 4A and 4B shows an example activity (squatting) performed by the subject and the corresponding metabolic cost measurement performed in the study.

FIGS. 5A-5E show detailed implementations of the flexible circuit employed in the study.

FIGS. 6A-6M depict the design and characterization of the example soft flexible bioelectronic system.

FIGS. 7A-7F shows mechanical testing of the example soft, flexible bioelectronic system.

FIGS. 7G-7I depict a breathability test across potential fabric materials employed in the study.

FIG. 7J-7Q show evaluation of the peeling forces in various humid conditions.

FIGS. 8A-8C shows the robotic ankle-foot-orthosis system and experimental setup employed in the study.

FIGS. 9A-9K shows the signal processing and analyses of the signals in the study, including analysis of the cardiac signals to determine the metabolic cost in the study.

FIG. 10A-10E depicts a training employed in the study to generate a trained machine learning model to predict metabolic cost in the study.

The components in the drawings are not necessarily to scale relative to each other. Like reference, numerals designate corresponding parts throughout the several views.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

Some references are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in a list. All references cited and discussed in this specification are incorporated herein by reference.

### Definitions

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to an “electrode” includes aspects having two or more such electrodes unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

### Example Systems

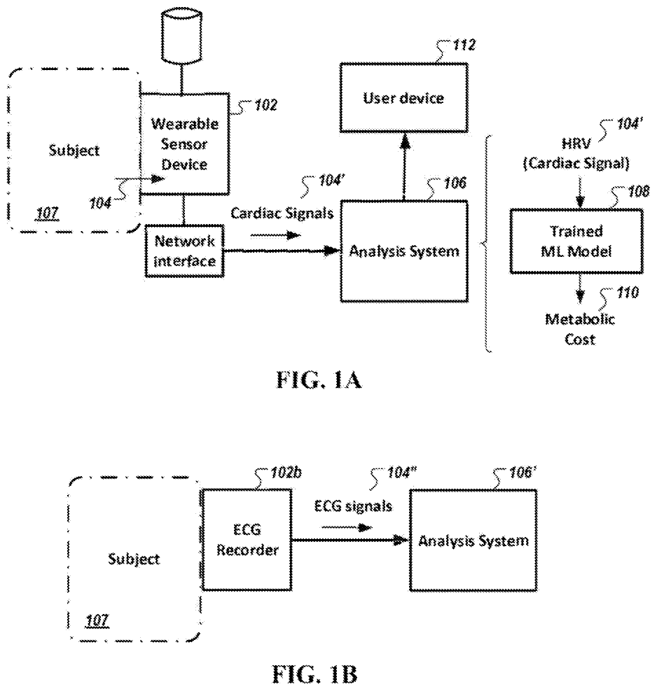

FIGS. 1A and 1B each shows an example device 100 (shown as 100a, 100b) configured to determine metabolic cost estimation from cardiac signals in accordance with an illustrative embodiment. FIG. 1A shows a wearable sensor device 102 configured to acquire the cardiac signals 104 in a portable manner and provide the signals 104 (shown as 104′) to an analysis system 106. The device 100a can be worn on the chest or torso of a subject 107, or patient. In some embodiments, the device 102 can be worn on the extremities, e.g., at the wrist, shoulder, arm, legs, and head. In some embodiments, the device 102 is a wearable device having an array of stretchable electrodes to be placed on the chest or back in proximity to the heart. In the example shown in FIGS. 1A, the analysis system 106 employs a trained machine learning model 108 configured to determine an estimation of metabolic cost 110 using the cardiac signal 104′ or a derived parameter, e.g., heart rate variability, determined from the cardiac signal 104′.

FIG. 1B shows the sensor device 102 (shown as “ECG recorder” 102b) configured to acquire and provide ECG signals (shown as 104″), as the cardiac signals, to the analysis system 106 (shown as 106′) to which the metabolic cost can be estimated. In some embodiments, the ECG recorder is an ECG equipment, e.g., a 12-lead device, 11-lead, 6-lead, 5-lead, etc.

In some embodiments, the analysis system 106 is an edge device configured to communicate with the wearable sensor device through a short-distance communication channel. In other embodiments, the analysis system 106 is implemented in cloud infrastructure to receive and determine the metabolic cost estimates and provide the determination to an edge computing device 112 (shown as “User device” 112). In some embodiments, the user device 112 is another wearable device such as a watch, smartphone, tablet, etc. In other embodiments, the user device 112 is acquisition device 102.

### Example Methods

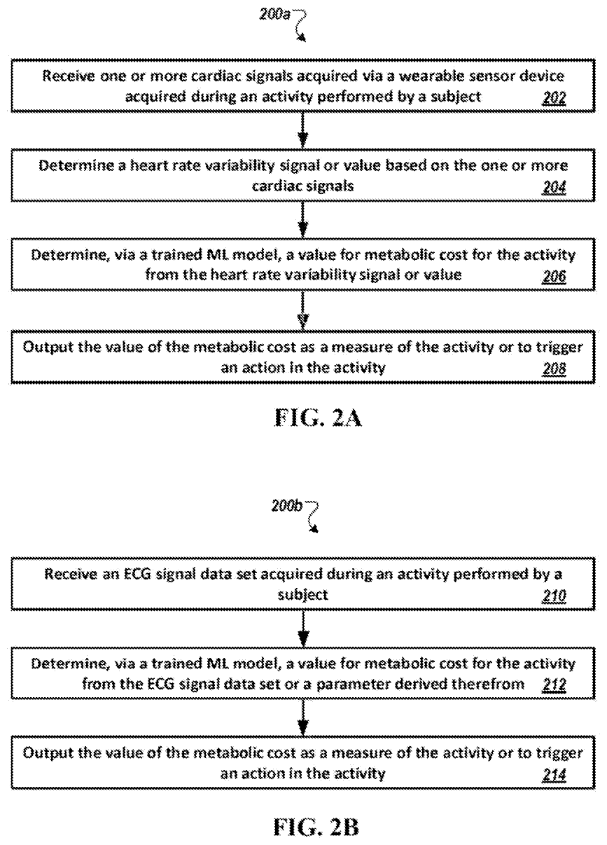

FIGS. 2A-2B show example methods 200 (shown as 200a, 200b), e.g., for using the example device 100a, 100b, among others, to calculate metabolic cost estimations from cardiac signals. In the example shown in FIG. 2A, method 200a includes receiving (202), e.g., at the analysis system, one or more cardiac signals (e.g., 104) acquired via a wearable sensor device (e.g., 102) acquired during an activity performed by a subject (e.g., 107). The signals (e.g., 104) may be measured from an array of stretchable electrodes, for example, in an embodiment. The method 200a then includes determining (204) a heart rate variability signal (e.g., a time series signal corresponding to the cardiac signal) or value (e.g., an average value for a window of the acquired cardiac signal). The method 200a then includes determining (206), via a trained ML model (e.g., 108), a value for metabolic cost (e.g., 110) for the activity from the heart rate variability signal or value. The method 200a then includes outputting (208), e.g., via a graphical user interface or a report, the value of the metabolic cost (e.g., 110) as a measure of the activity or to trigger an action in the activity.

The activity can be a US Bureau of Labor Statistics-defined activity or labor research-defined activity, e.g., labor tasks such as construction-associated labor activities, PV installation-associated labor activities, warehouse operations, material moving, fishing-associated labor activities, farming-associated labor activities, forestry-associated labor activities, nursing, military, grounds cleaning, office labor (e.g., accountant, lawyers, office worker), etc. In some embodiments, the activity is associated with those in a clinical setting, e.g., rehabilitation. In some embodiments, the activity is associated with those in a sport evaluation, e.g., performance analysis. In some embodiments, the activity is an activity described in any one of the provided references.

In the example shown in FIG. 2B, method 200b includes receiving (210) an ECG signal data set (e.g., 104″) acquired during an activity performed by a subject. The method 200b then includes determining (212), via a trained ML model (e.g., 108), a value for metabolic cost (e.g., 110) for the activity from the acquired ECG signal data set (e.g., 104″). The method 200b then includes outputting (214), e.g., via a graphical user interface or a report, the value of the metabolic cost (e.g., 110) as a measure of the activity or to trigger an action in the activity for a moving calorimetric test. An action can for example be a change in intensity (e.g., increase or reduction) of the activity. In some embodiments, the activity is running/treadmill activity, pulmonary test activity, etc. In some embodiments, the action includes an alert on the metabolic cost for the activity being above or below a pre-defined threshold. The threshold may be variable based on a baseline measurement of the metabolic cost.

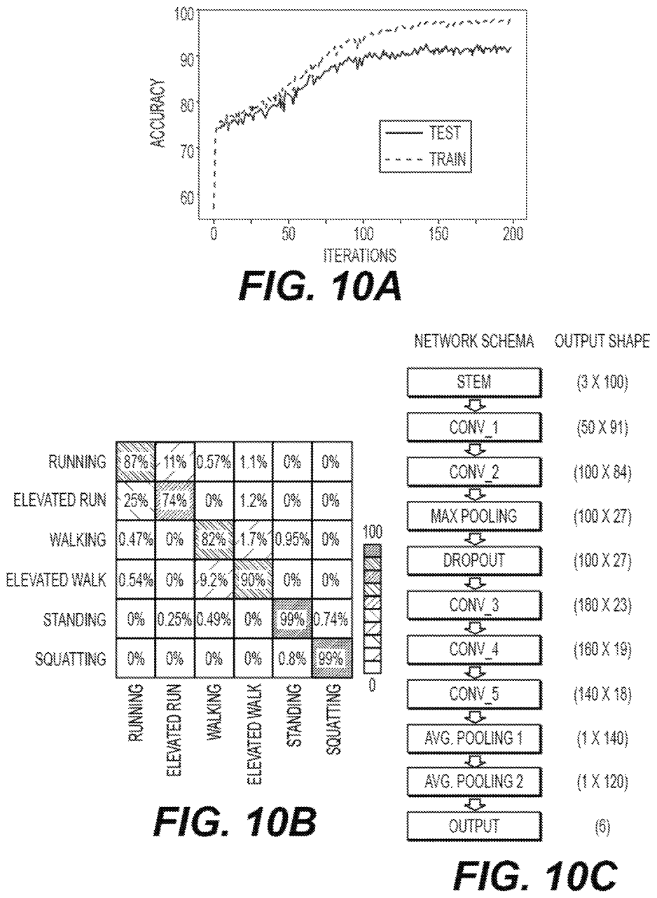

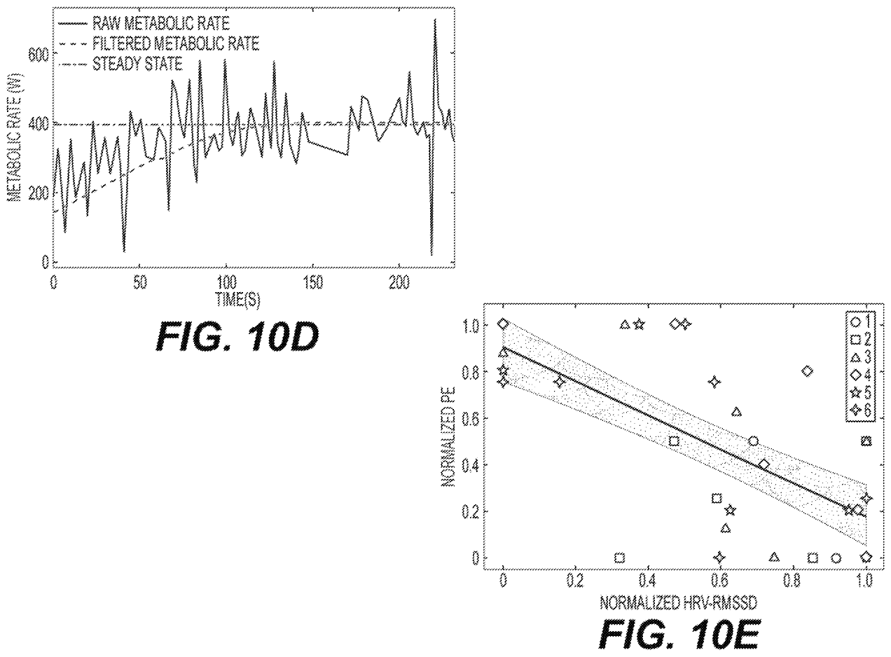

Example implementations of the training method are provided in relation to FIGS. 10A-10E, among others.

### Experimental Results and Additional Examples

A study was conducted to develop a wearable bioelectronic system having a soft biopatch and a machine-learning algorithm to estimate metabolic costs and physical effort. The wearable bioelectronic system was employed in conjunction with an ankle-foot exoskeleton to evaluate and/or quantify metabolic cost reduction from the ankle-foot exoskeleton. It was observed that the miniaturized, all-in-one wearable biopatch can measure highly accurate metabolic rates to replace the existing bulky, heavy, and cumbersome tools. The soft biopatch can detect motion hardness, cognitive effort, and physical effort as metabolic costs and energy expenditure, e.g., in the exosuit study, and is contemplated to be broadly applicable to any physical activity, particularly, labor-associated activities. The wearable bioelectronic system is a skin-conformal device can directly contact the skin to provide a high-quality recording of cardiac signal (ECG) and HRV-RMSSD. Unlike conventional mask-based calorimetry devices, the instant wearable system employs soft, flexible bioelectronics (SFB) to provide a high SNR (>25 dB) on different activities (walking, running, and squatting) and high Pearson R correlation (−0.758, p-value: 1.2e-7) [33] to steady-state metabolic costs. The portable biopatch can provide a measure of muscle activities, temperature, and stress levels, providing an insight into real-time human-in-the-loop optimization, e.g., while used in combination with a biosuit.

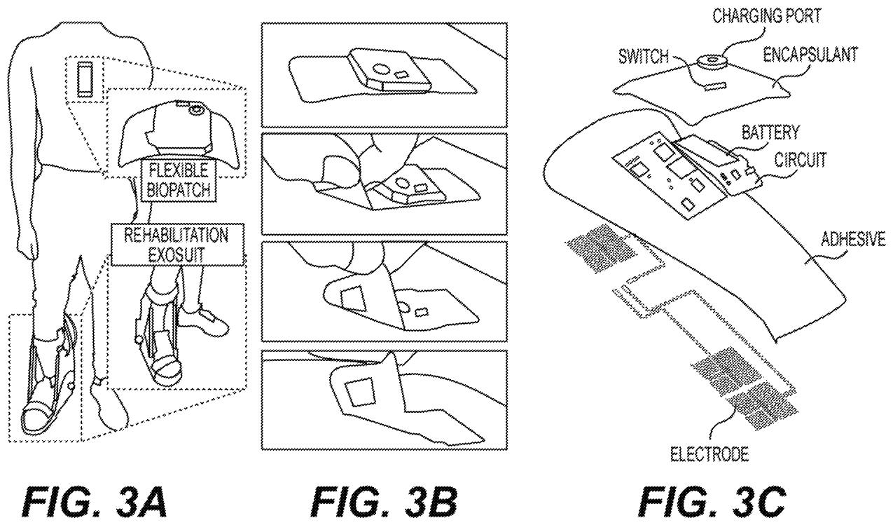

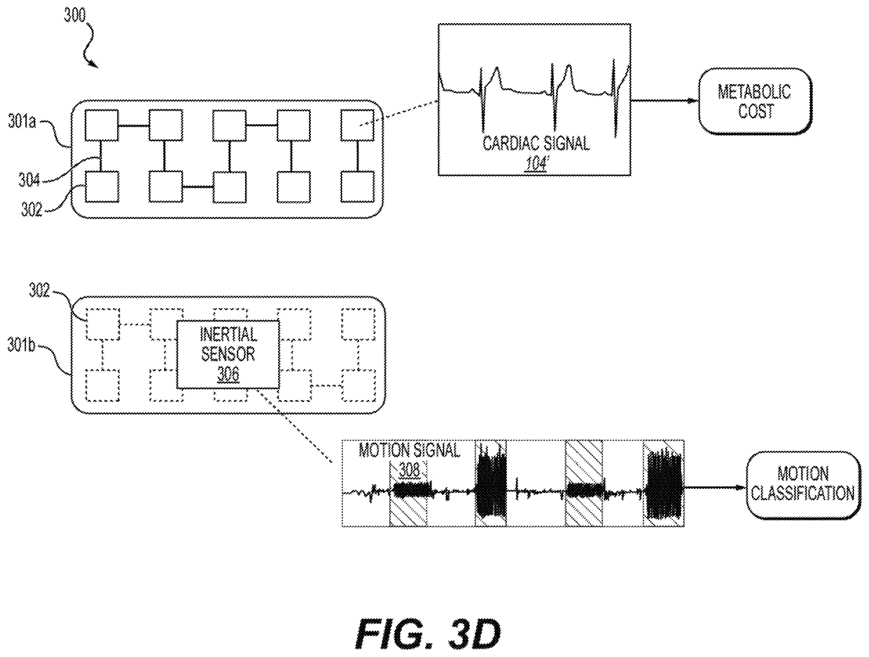

Example wearable bioelectronic system. FIGS. 3A-3E show an example soft flexible bioelectronic system (SFB), e.g., as a wearable sensor device 100, employed in a study that used an ankle-foot-orthosis (AFO) exoskeleton to estimate metabolic costs and physical effort. To monitor the ECG and determine HRV-RMSSD during squatting and compare with metabolic cost, the study attached the SFB to a subject's sternum. The attachment to the subject's sternum can reduce chest muscle motion artifacts that can cause misinterpretation of bio-signals during squatting. The location of SFB in the sternum provided a larger motion range with less motion artifacts and greater compatibility with other potential electronics that measure other bio-signals along with AFO.

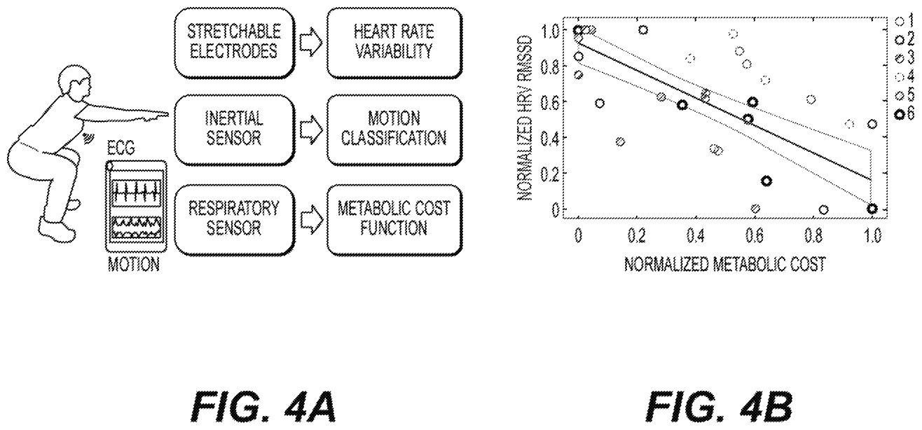

FIG. 3A shows a photo of a subject wearing an SFB and an AFO. FIG. 3B shows a photos of the skin-mounted SFB and its conformal lamination and soft contact to the skin. In FIG. 3B, the scale bar=1 cm. FIG. 4C shows a CAD exploded view of the SFB with its integrated components. The scale bar=1 cm. In FIG. 3C, the SFB is shown to have a multilayered circuit architecture that includes 1) a soldered flexible printed circuit board (fPCB) and power supply, 2) a battery assembly with magnetic charging port and switch, and 3) a micro-manufactured laser patterned gold bipolar and a reference electrode on a flexible and stretchable substrate (9907T, 3M). FIG. 3D shows a functional diagram of the SFB as a wearable sensor device. In FIG. 3D, the SFB included a substrate, as a flexible adhesive patch, having a first side 301a and second side 301b. The substrate on the first side 301a includes an array of stretchable electrodes 302 joined by a plurality of interconnectors 304 that are configured to be in contact with the skin of the subject (e.g., 107) to provide a measure of cardiac signals (e.g., 104′) to be used to determine metabolic cost estimation. In FIG. 3B, the second side 301b shows the electrode 302 as a dotted outline, as the electrodes are not visible from the second side of the device. The second side 301b includes an inertial sensor 306 configured to output a motion signal 308, that can be used for motion classification. The study contemplated the inclusion of other sensors, e.g., temperature, magnetic-based sensors, and acoustic sensors.

FIG. 4A shows an example activity (squatting) performed of the subject and the corresponding sensors and output of the analysis. Wireless transmission was performed for the ECG, angular velocity, and 3-axis acceleration data through a Bluetooth-low-energy-enabled circuit to the android phone during squatting (left). During the activity, the subjects used the AFO exoskeleton. Other activities included squatting and walking-running. During the activity, ECG, HR, and metabolic costs were measured and analyzed during each session using a trained machine learning system. HRV-RMSSD was calculated from filtered HR, and the metabolic cost was determined by indirect respiratory calorimetry.

FIG. 4B shows a plot showing the determined relationship between normalized HRV-RMSSD from the SFB and normalized metabolic cost from a calorimetric respiratory mask. In FIG. 4B, the normalized metabolic cost and normalized HRV-RMSSD results are shown to have a Pearson R correlation of −0.758 with a p-value of 1.2e-7, indicating a strong negative correlation between calorimetry and HRV.

Preparation of a soft flexible biopatch: This study developed an SFB with multiple electronic components, wireless flexible printed circuit board (fPCB), and stretchable electrodes. The fPCB provides mechanical flexibility and low Young's modulus that can provide suitable conformal contact with the skin. In the study, the fPCB included a main circuit board and a power supply circuit board. The mainboard is 25 mm×14 mm in size, having a double copper layer with a 12.7 μm polyimide separation and immersion gold surface finish. The power supply board is 18 mm×10.2 mm, having a double copper layer with the same separation and surface finish resulting in an overall thickness of 0.5 mm. Multiple fPCBs reduce the system's rigidity by providing more freedom to bend and less volume by stacking the main board on top of the power supply board.

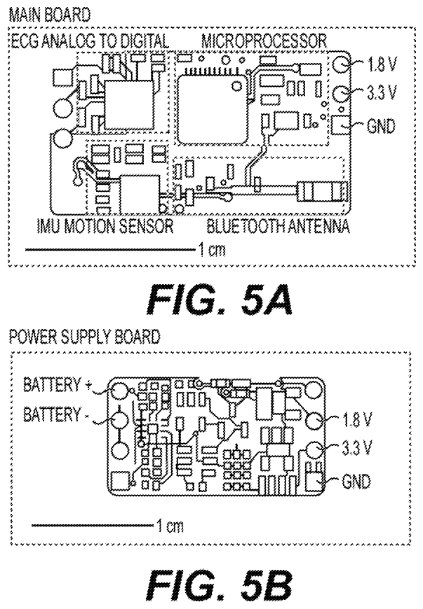

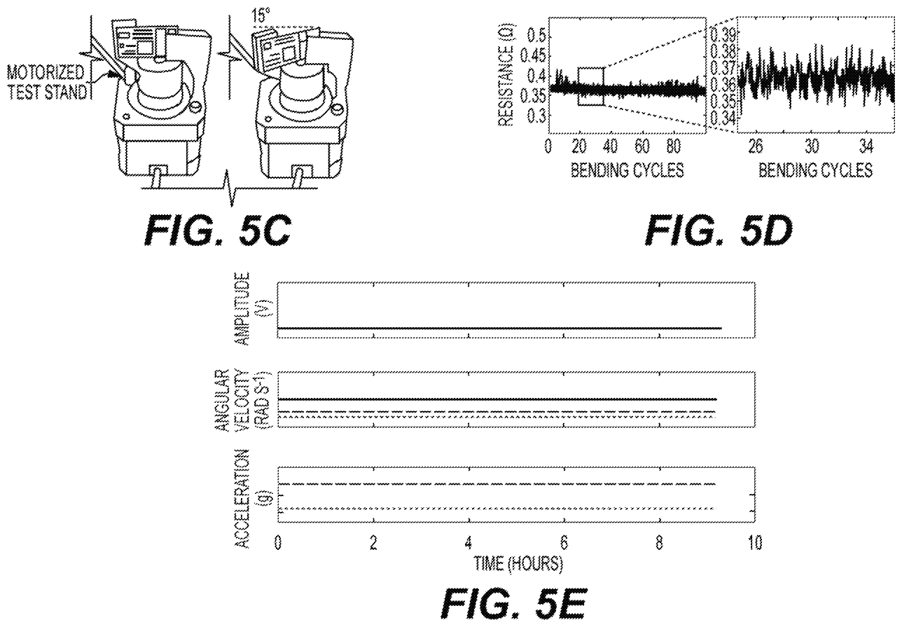

FIGS. 5A-5B show detail implementations of the flexible circuit employed in a study. The circuit acquired cardiac signals for determination of metabolic cost in accordance with an illustrative embodiment. FIG. 5A shows the main board. FIG. 5B shows the power supply board that provides power to the main board. In FIG. 5A, the main board included 4 main circuit components: ECG analog-to-digital converter circuit (ADS1292, Texas Instruments), microprocessor circuit (NRF 52832, Nordic), IMU motion sensor circuit (IMU20948, InvenSense), and Bluetooth Antenna circuit. The analog-to-digital converter was configured to receive raw voltage from bipolar electrodes to be placed in contact with the skin and convert the analog signal into a digital signal along with the IMU sensor. The digital signals were received at the microprocessor and transmitted through the high-frequency (~2.4 GHz) low-power Bluetooth antenna. The power board had a 3.7-V input from a battery (3.7 V, 40 mAh, 1.13 g) and provided two different power lines via a 1.8 V regulator (S1318A18, ABLIC) and a 3.3 V regulator (S1318A33, ABLIC) to power the mainboard. TABLE 1 shows the components of the fPCB mainboard and power supply board.

TABLE 1

| Component | Description | Value | | Part Number |
|---|---|---|---|---|
| A1 | 2.4 GHz | n/a | | 2450AT18A100 |
| | antenna | | | |
| X1 | 32 MHz | n/a | | ECS-320-8-37CKM |
| | oscillator | | | |
| X2 | 32 kHz | n/a | | ECG-327-9-12-TR |
| | oscillator | | | |
| U1 | 1.8 V regulator | n/a | | S-1318A18-M5T1U4 |
| U2 | 3.3 V regulator | n/a | | S-1318A33-M5T1U4 |
| U3 | microcontroller | n/a | | NRF52832-QFAA-R |
| IC1 | biopotential | n/a | | ADS1292IRSMT |
| | AFE | | | |
| IC2 | motion sensor | n/a | | ICM20948-INSE- |
| | | | | QFN |
| D1 | Schottky diode | n/a | | 641-1285-1-ND |
| D2, D3 | LED | n/a | | |
| C1, C4 | 0402 capacitor | 4.7 | nF | |
| C2 | 0402 capacitor | 1 | nF | |
| C3, C6, C9, | 0402 capacitor | 1 | μF | |
| C27, C30 | | | | |
| C5, C8, | 0402 capacitor | 100 | nF | |
| C10, C16, | | | | |
| C19, C21, | | | | |
| C23, C24, | | | | |
| C25, C28, | | | | |
| C31 | | | | |
| C7, C26, | 0402 capacitor | 10 | μF | |
| C29 | | | | |
| C11 | 0402 capacitor | 1 | μF TANT | |
| | TANT | | | |
| C12, C13, | 0402 capacitor | 12 | pF | |
| C14, C15 | | | | |
| C17 | 0402 capacitor | 100 | pF | |
| C18 | 0603 capacitor | 1 | μF | |
| C20 | 0402 capacitor | 4.7 | μ F | |
| C22 | 0402 capacitor | 1 | pF | |
| R1, R2 | 0402 resistor | 30 | kΩ | |
| R3, R4 | 0402 resistor | 422 | Ω | |
| R5, R6, | 0402 resistor | 10 | kΩ | |
| R7 | | | | |
| L1 | 0402 inductor | 15 | nH | |
| L2 | 0402 inductor | 10 | μH | |
| L3 | 0402 inductor | 3.9 | nH | |
| L4 | 0402 inductor | 2.7 | nH | |
| L5 | 0402 inductor | 1 | μH | |

| | | |
|---|---|---|
| B1 | 40 mAh Li-ion | n/a |
| | battery | |

Mechanical reliability: To conform the fPCB to the skin curvatures, the study designed the fPCB to bend up to at least 15°. To test the flexibility, the fPCB was tested with a cyclic bending test. FIGS. 5C-5D show mechanical testing via bending test on the wearable sensor device. FIG. 5C shows a photo of the experimental bending analysis setup. FIG. 5D shows resistance across the two furthest points of the fPCB with 100 cyclic loadings. FIG. 5E shows the maximum operation of the circuit in battery lifetime with 40 mAh.

In the test, a fully soldered mainboard was mounted on a custom-built motorized test stand (FIG. 5C), and resistance between the two furthest ground pads was measured during cyclic bending of 15° to −15° for 100 cycles. Overall, the study observed no shifting of resistance or sudden failure. The fPCB provided a consistent fluctuation range between 0.35Ω and 0.38Ω, showing the sufficient quality of fPCB to be used on human skin (FIG. 5D).

Human subject study: The study conducted a set of squatting, walking, and running evaluations with fabricated SFB, AFO, and mask indirect calorimetry. The human pilot study included six participants: 4 males and 2 females, weight between 57 kg-91 kg, height between 157-185 cm, and 21-57 years of age. In the experiments, each squatting phase was done for 4 minutes with the subject squatting for 2 s (1 s descending and 1 s ascending) and standing for 6 s before and after squatting, followed by walking and running with different magnitudes of elevation. After each condition, the subjects were asked to rest for 12 minutes with a ratio of 1:3 to squatting time [34]. The AFO emulator was worn on the subject's dominant limb during squatting. All squatting sessions were controlled by a metronome and timer display on a screen. The devices acted as audio and visual cues for the squat and rest. The subjects were asked to perform a full squat or the deepest squat possible with a distance between ankles approximately shoulder-width apart [35], [36]. The squat posture and leg position were determined prior to the start of the study based on the subject's capability and biomechanical range and are fixed for the complete study.

Classification Model: The study employed pre-processing that first segmented the 3-axis acceleration data of six subjects segmented into 0.5 second increments. Similar pre-processing can be employed in an inference prodcution system. The segmented data were labeled for six conditions (running, elevated running, walking, elevated walking, standing, and squatting) and then split up in 80:20 for train and testing, respectively. The set was then loaded to the pytorch [37] dataloader module, and training data order was randomized for each training epoch. To perform activity classification, three convolutional layers were employed in the training of the model, followed by the max-pooling layer and dropout layer. Three more convolution layers then followed these layers. Finally, a fully connected layer with 6 outputs is used. For each layer, the output is activated using reLU activation. For the final layer, the activation is soft-max. The convolution layer has one stride, and the training was performed in the batch of 50 with cross-entropy loss and Adam optimizer with a learning rate of 0.001. The model and training were built using pytorch library, and each iteration training time was 15 seconds using GPU (Geforce RTX 2060 super, NVIDIA). During the training process, the training and test loss and accuracy for each epoch were recorded. The model with the best test accuracy was used to report results.

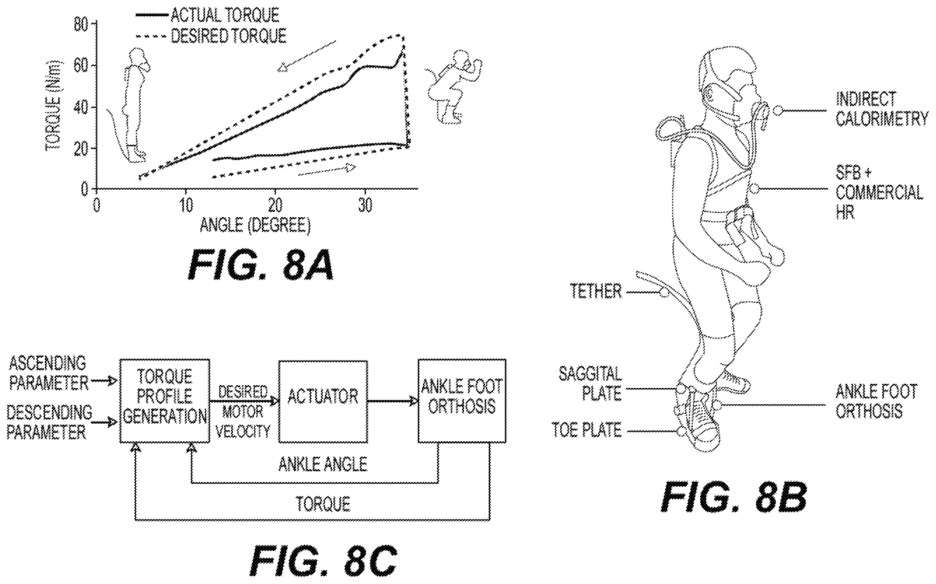

Robotic AFO setup: The setup for the squatting assistance used an AFO emulator, composed of high and low-level controllers and actuators. The high-level controller generated additional desired torque using an impedance curve shown in FIG. 8A. The AFO had ascending and descending parameters to control the torque profile. The ankle angle and applied torque were measured using a load cell and the rotary magnetic encoder. Using the desired torque, the low-level controller conducted a torque control and commanded control input, desired velocity, to the servo actuator (Humotech®). The power and signal were transmitted through wires to the AFO end-effector.

### Results and Discussion

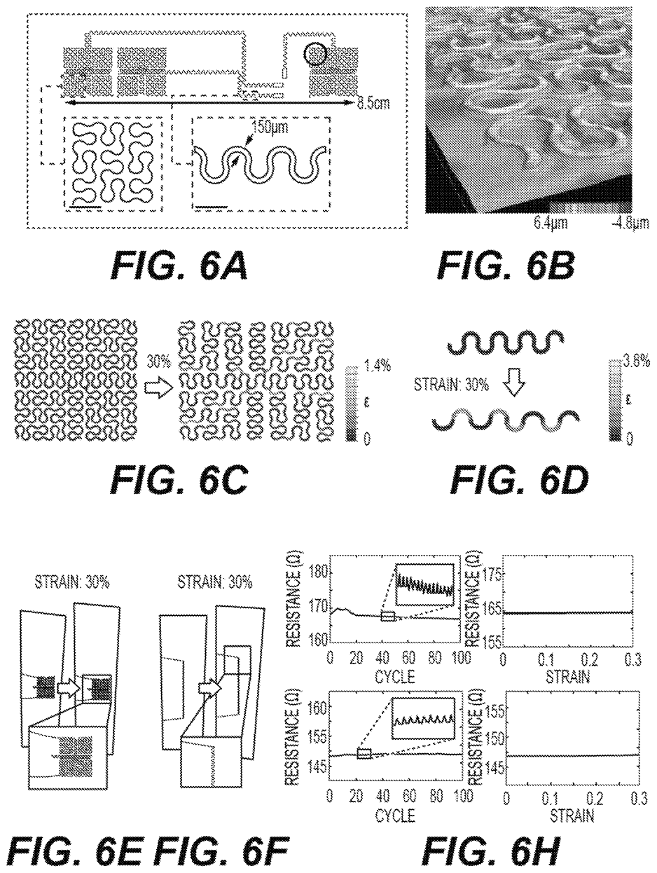

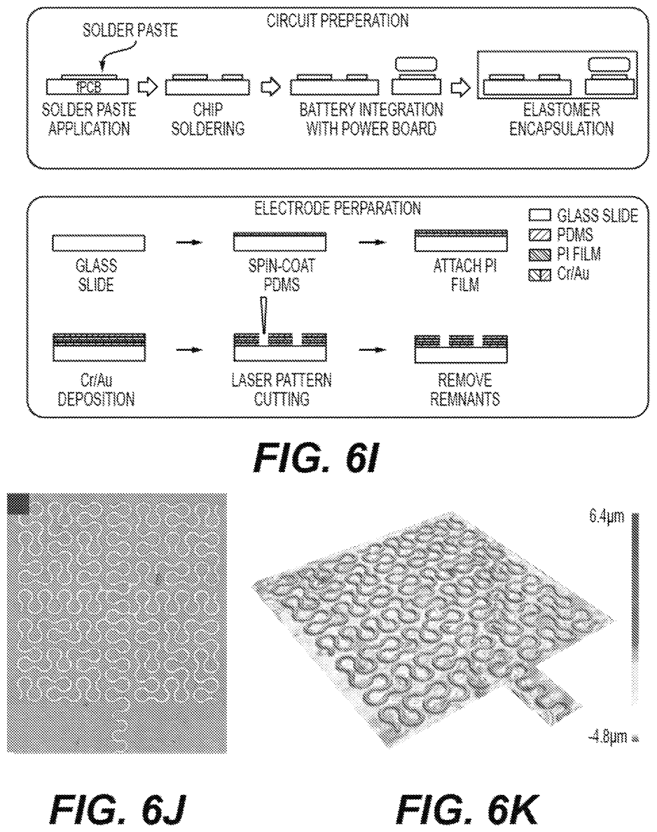

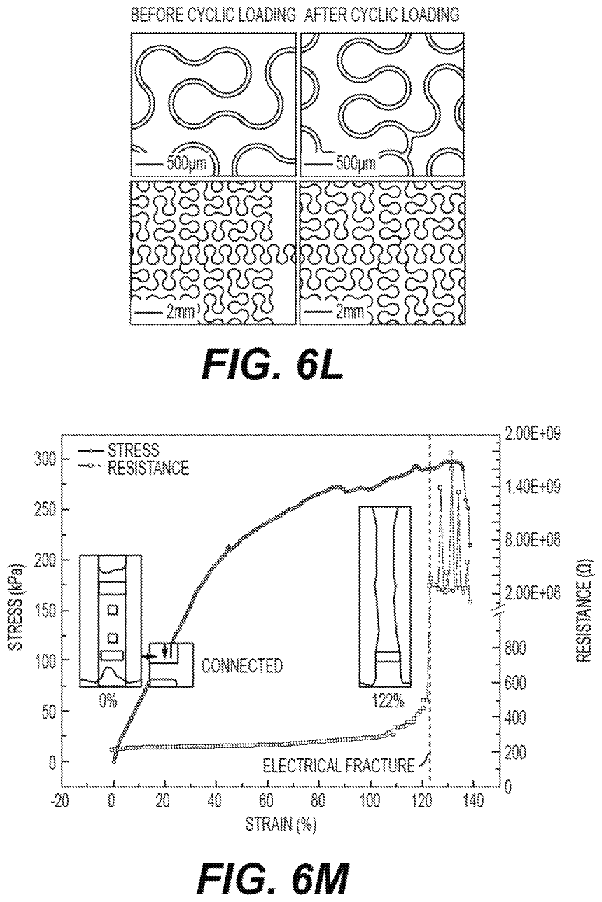

Design and characterization of an SFB: FIGS. 6A-6M depict the design and characterization of the example soft flexible bioelectronic system.

The study used electrode fabrication methods utilizing a high precision micro-laser machine (Femtosecond Laser Micro-Machining System, OPTEC) to provide electrode manufacturing of large batches with minimized cleanroom use and rapid prototyping without photolithography. To fabricate electrodes, polydimethylsiloxane (PDMS) was spun-coated in glass slides, followed by chromium and gold deposition of 10 nm and 200 nm, respectively. The gold-deposited slide was then patterned with a micro-machining system.

FIGS. 6A-6H shows the dimensional details and physical characteristics of electrodes and interconnectors. Specifically, FIG. 6A shows an illustration showing the dimensions of an array of stretchable electrodes and interconnectors. Scale bar: 1 cm (main), 2 mm (inset-left), and 1 mm (inset-right). FIG. 6B shows a 3D profilometer image of the stretchable electrode in FIG. 6A. FIGS. 6C-6D show computation modeling results showing the electrode's stretchability (FIG. 6C) and the interconnector's stretchability (FIG. 6D) under 30% tensile strain. FIGS. 6E-6F show experimental validation of the stretchability of an electrode (FIG. 6E) and interconnector (FIG. 6F) during cyclic loading with 30% strain. Scale bar: 1 cm. FIG. 6G shows measurements of the electrical resistance of the electrode during cyclic loading (100 cycles; left) and 30% strain (right). FIG. 6H shows measurements of electrical resistance of the interconnector during cyclic loading (100 cycles; left) and 30% strain (right).

The bipolar electrode system for SFB was made of three identical electrodes having patterns with 8.5 cm long and 7.5 cm center-to-center distance from positive to negative to provide good ECG signal quality. FIG. 6I depicts schematics of the electrode fabrication with laser cutting and fPCB preparation. Specifically, FIG. 6I shows the circuit board assembly (top) and electrode fabrication (bottom) details. Each electrode pattern was guided to an appropriate location via serpentine interconnects for easier construction of the SFB. The electrode pattern had a width of 150 μm (FIG. 6A) and a total thickness of 8 μm (FIG. 6B).

Profilometry. Profilometry measurements were made using a profilometer (VK-X3000 3D Profilometer, Keyence) and confirm sufficient flexibility and stretchability to maintain conformal contact with the skin.

FIGS. 6J-6K depict photos of a stretchable electrode and the corresponding profile image using the Keyence VK-X3000 3D surface profiler. FIG. 6J shows an optical image of a single laser-cut electrode pattern. Specifically, FIGS. 6J-6K show details of profilometric data with a top-down optical photo of a single laser-patterned electrode (left) and a profilo-metric illustration of the electrode with color-matching height variance (right). FIG. 6K shows a scanned profile of the electrode in FIG. 6J. FIG. 6L depicts microscopic photos of electrodes before and after cyclic loading for the stretchability test. Electrodes show good adhesion to the substrate before cyclic loading (left) and still show no delamination after cyclic loading (right). FIG. 6M depicts a stress-strain curve with an SFB during an elongation test with electrical resistance measurement. The stretchability of electrodes and interconnects were computationally calculated in finite element analysis (FEA; Abaqus, Dassault Systèmes) with 30% uniaxial elongation to estimate its durability under skin stretch during squatting (FIGS. 6C-6D). Both electrode and interconnect FEA results were observed to be under 4% maximum von Mises stress with 30% uniaxial elongation. The result show sufficient stress for gold-deposited layers.

For experimental validation, electrodes and interconnects were mounted on a soft substrate (9907T, 3M) and loaded on cyclic uniaxial stretching. FIGS. 6E-6F show the resistance between the two furthest points for an electrode and interconnector, respectively. FIG. 6G shows details of resistance change per load cycle. In FIG. 6G, despite a slight shift of resistance over time, the resistance change within a single cycle was observed to be less than 0.05Ω, corresponding to less than 0.1% change. The electrode test showed little to no difference with an increase in the strain, which portrays the durability of SFB against skin elongation during squatting. The resistance change against uniaxial cyclic loading is minimal, less than 2Ω. Similarly, the interconnector (FIG. 6H) shows little to no change with an increase in the strain, which portrays the durability of SFB against skin elongation during squatting.

FIG. 6L shows a zoomed-in optical image taken before cyclic loading and after to confirm little to no delamination via microscope. An epidermal electronics should withhold 30% of strain due to the nature of skin elongation limitation [23]. FIG. 6M presents an SFB's stress-strain curve until the fracture point; the SFB shows an electric response up to 50% elongation before turning into a plastic response and finally fracturing at 123% of extension. The slope of the stress-strain curve shows Young's modulus of 500 kPa, showing the SFBs having sufficient conformal contact on the subject's chest under skin elongation.

Fabrication. Patterned electrodes were transferred to a stretchable substrate with water-soluble tape to fabricate the device. The mainboard was stacked on top of the power board and connected with copper wires for corresponding power line pads. The battery assembly was put aside from the fPCB stack but placed in a position where the high-frequency antenna is not affected by the conductance of the battery system. The powered board system was then put on a biocompatible soft stretchable layer (Ecoflex™ Gel, Smooth-On) to reduce stress on skin attachment, followed by placement on top of the substrate. The analog inputs were connected to the corresponding electrode pattern with asymmetric conductive films (ACFs) using fast-drying silver paint (Leitsilber Conductive Silver, Ted Pella). The silver paint with ACFs was cured at 60° C. for 1 h to secure the electric connection fully. The attached ACFs were routed directly to corresponding electrode pads through the gap created from a small cut. Lastly, a soft elastomer (Ecoflex™ 30, Smooth-On) encapsulated the device to protect the circuit electronically and mechanically.

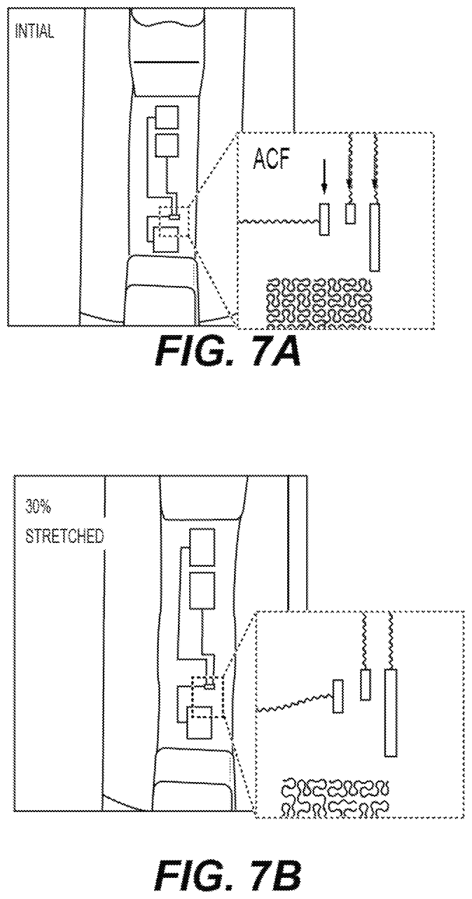

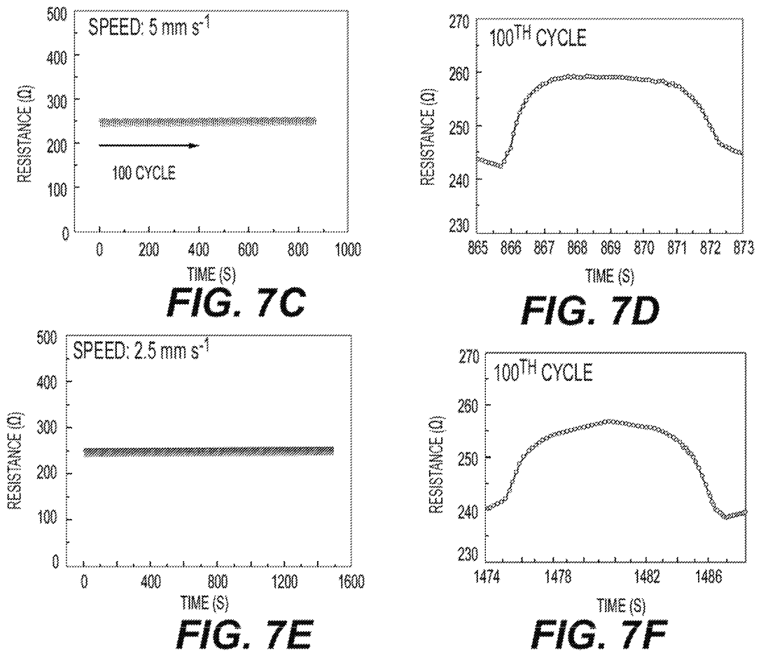

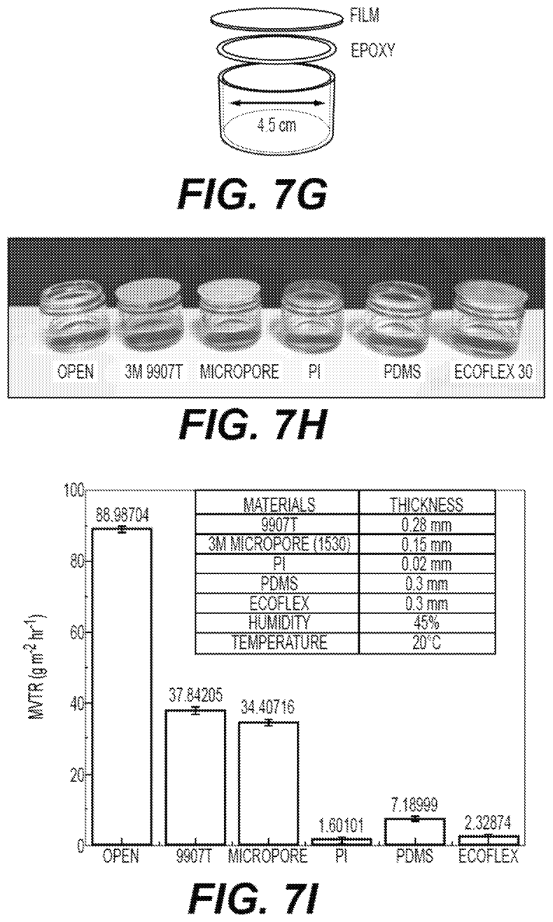

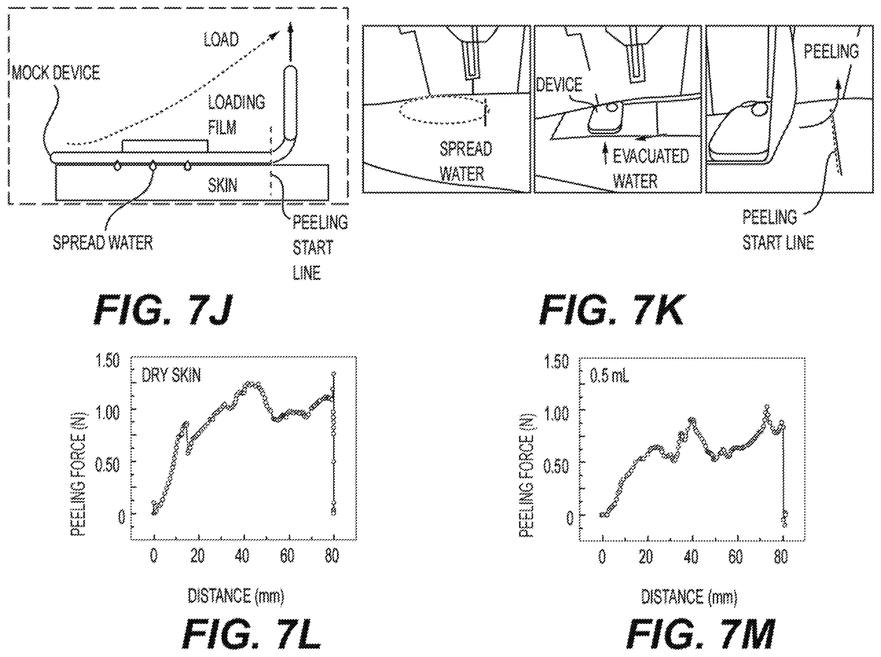

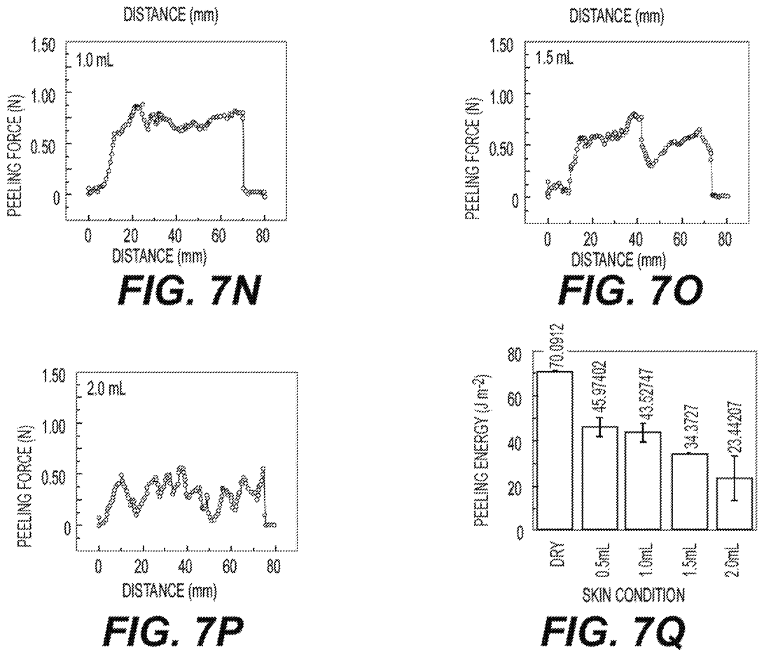

Stretchability test. FIGS. 7A-7F shows additional device stretchability tests with different stretching speeds, with a detailed view of the ACF connection and stretchability test. Two different stretching speeds were used to elongate the SFB at 5 mm s⁻¹. Similarly, the stretching test result of 2.5 mm s⁻¹ is shown. For both cases, the whole device had minimal resistance change of less than 1% overall, and the final cycle proves its ability to return the original value after 30% elongation.

Breathability Evaluation. FIGS. 7G-7I shows a comparison of the breathability of the fabric used in the design of SFB with other potential fabrics and materials. FIGS. 7G-7H illustrate the experimental setup of the breathability test calculating the moisture vapor transmission rate (MVTR) by measuring water evaporation weight through each material for 72 hours. In FIG. 7I, the MVTR result indicated that the 3M 9907T has superior breathability compared to other elastomers, such as PDMS, Ecoflex, and micropore layers.

FIG. 7A shows a photo of the experimental setup of the SFB ACF connection. FIG. 7B shows a photo of the ACF connection after 30% elongation cyclic test. FIG. 7C shows a graph of SFB electrode-to-electrode resistance along 100 cyclic loading of 30% elongation with 5 mm s⁻¹ stretching speed. FIG. 7D shows a zoomed-in graph of the last 100th cycle of the elongation test with 5 mm s⁻¹ stretching speed. FIG. 7E shows a graph of SFB electrode-to-electrode resistance along 100 cyclic loading of 30% elongation with enhanced 2.5 mm s⁻¹ stretching speed. FIG. 7F shows a zoomed-in graph of the last 100th cycle of the elongation test with a 2.5 mm s⁻¹ stretching speed.

Peeling force/delamination evaluation. FIGS. 7G-7I depict a breathability test across potential fabric materials. FIG. 7G shows an illustration of the test jar setup. FIG. 7H shows a breathability testing photo of 5 different materials, including control, 3M 9907T, Micropore, PI sheet, PDMS, and Ecoflex™ 00-30. FIG. 7I shows the result of the breathability test with the thickness chart in the inset.

FIG. 7J-7Q show evaluation of the 907T's peeling forces in various humid conditions. Specifically, FIGS. 7J-7Q depict a peeling force test from the skin. FIG. 7J shows an illustrated view of the SFB peeling force test from the skin. FIG. 7K shows a series of pictures representing the peeling force steps. FIGS. 7L-7P show peeling force result graphs at different skin conditions with hydration level of dry skin, 0.5 mL, 1.0 mL, 1.5 mL, and 2.0 mL, respectively. FIG. 7Q shows a peeling energy comparison among different skin hydration levels.

In the evaluation, the sample device was attached to the arm with a force transducer pulling directly upward. The fabric maintained up to 1N of peeling force with 1.0 ml of water drop on the arm, which shows sufficient attachability to withhold its position against delamination [24]. FIGS. 7J-7Q show the detailed peeling energy calculation with peeling energy of 70 J m⁻² on dry skin and degradation to 40 J m⁻² with water drop.

Robotic ankle-foot system and metabolic cost estimation: This study used a two-degree freedom ankle-foot orthosis end-effector with an active plantarflexion utilizing a tethered emulator system for the squatting assistance.

FIGS. 8A-8C provide a description of the exosuit. FIG. 8A shows a comparison between the actual torque (blue) and desired torque (orange) according to ankle angle. The angle-torque relationship compares the torque trajectory commanded to the low-level controller and the corresponding torque observed at the exosuit. The exosuit included off-board actuator that is configured to transmit mechanical power via two Bowden cable tethers attached to the orthosis. Ankle range of motion can be provided between −80° and 50° in plantarflexion and −20° to 20° in inversion-eversion, with respect to the neutral standing position. Plantarflexion occurs when both toes rotate in the same direction. The squatting trajectory was designed to assist the subject both while ascending (moving up) and descending (moving down) (FIG. 8A). During these phases, the assistance or desired torque was proportional to the ankle angle, where the proportional constant is changed based on the condition. The controller was previously tested and has also been used to personalize the assistance using human-in-the-loop optimization (FIG. 8B) [4], [11].

FIG. 8C shows an illustration of the experiment setup, where the subject wears the AFO exosuit on the right foot, the SFB measuring ECG, and the mask-based calorimetry device on the face. FIG. 8C shows a detailed system architecture for the mid-level and low-level controllers. FIG. 8C also illustrates the whole system of SFB and AFO with indirect mask calorimetry (K5, Cosmed). The AFO and SFB are attached to the participants' sternum after cleaning with isopropyl alcohol and proper drying. The powered SFB was connected to a nearby android device to record raw ECG and motion signals. In addition, respiratory measures are recorded to determine the metabolic cost of squatting. The indirect respiratory calorimetry measures oxygen intake and carbon dioxide outtake to calculate the estimated metabolic cost from Eq. (1).

$$\\ \begin{matrix}Estimated Metabolic Cost = \frac{16.58}{60} \times {Volume}_{{O}_{2}} + \frac{4.51}{60} \times {Volume}_{{CO}_{2}} & ( Eq . 1 )\end{matrix}$$

The estimated metabolic costs are divided by the subject's body weight and then normalized to compare with HRV-RMSSD. The AFO emulator is worn on the subject's dominant limb during squatting. The participants are required to maintain a standing position for the baseline condition. They are asked to squat under six conditions in random order, including no power AFO condition and no assist condition by AFO. After squatting sessions, the participants are asked to remove AFO and perform walking and running. The orders of walking and running are randomized for every subject. In contrast to squatting, resting times for walking and running are 3 min and rest.

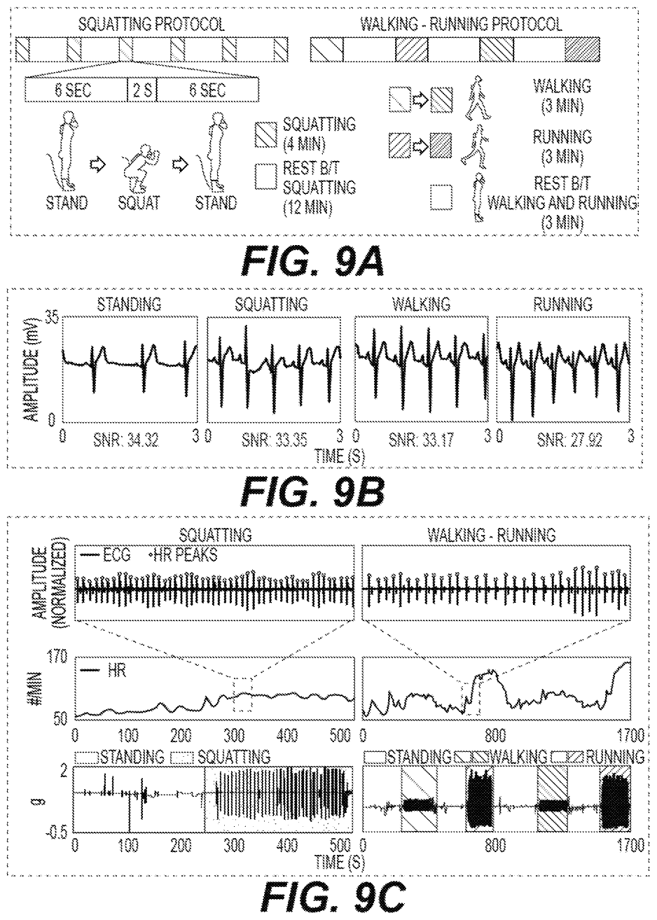

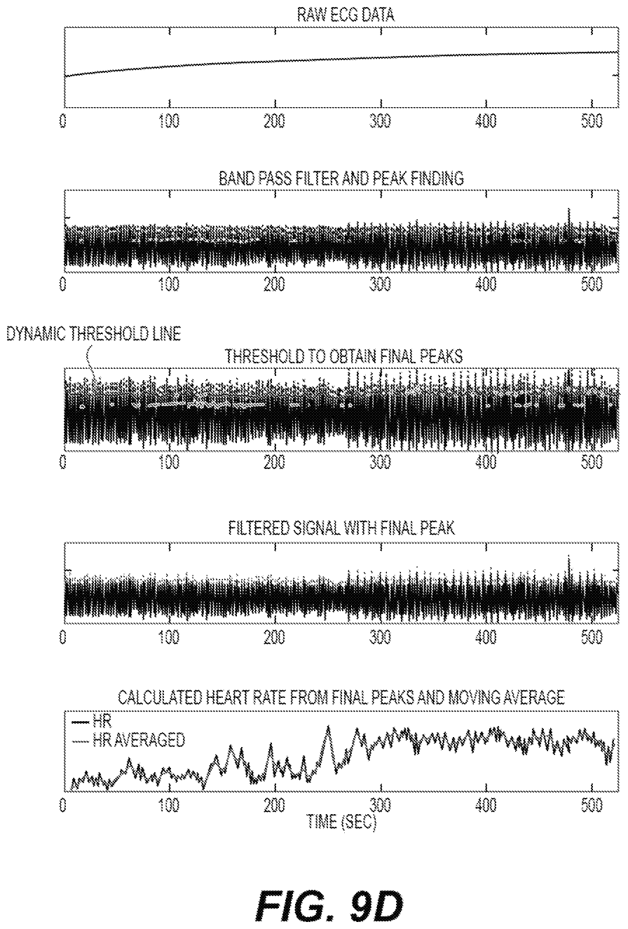

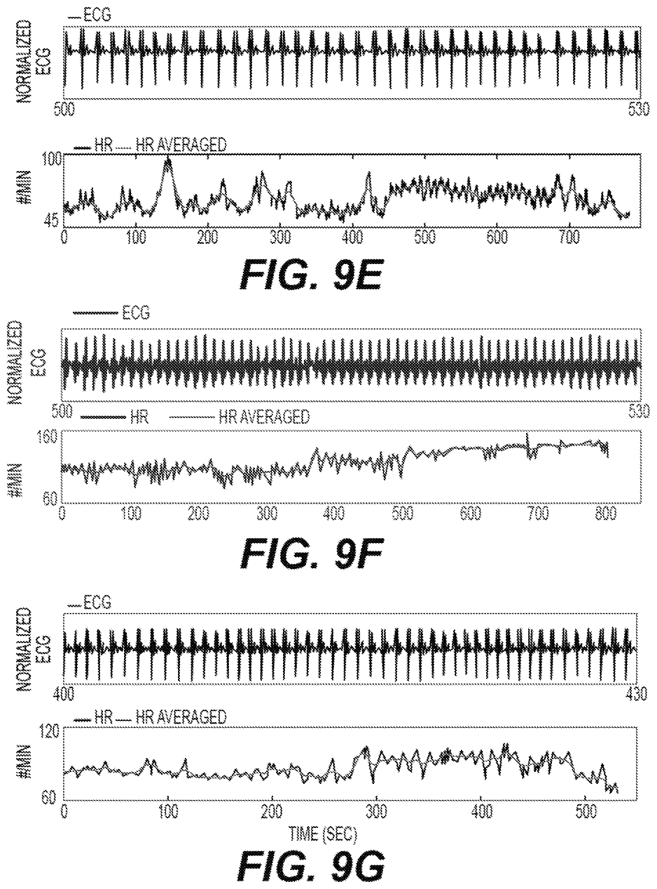

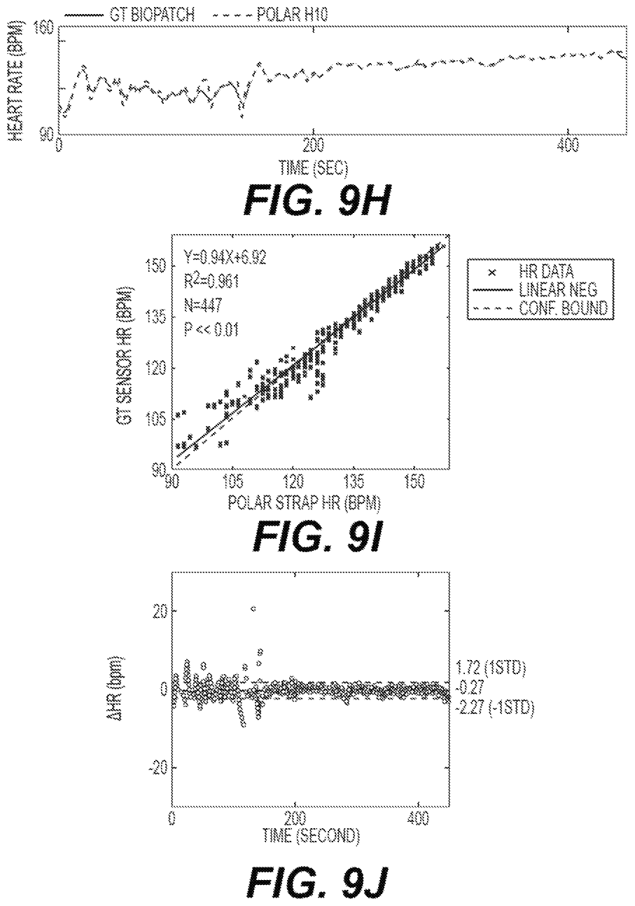

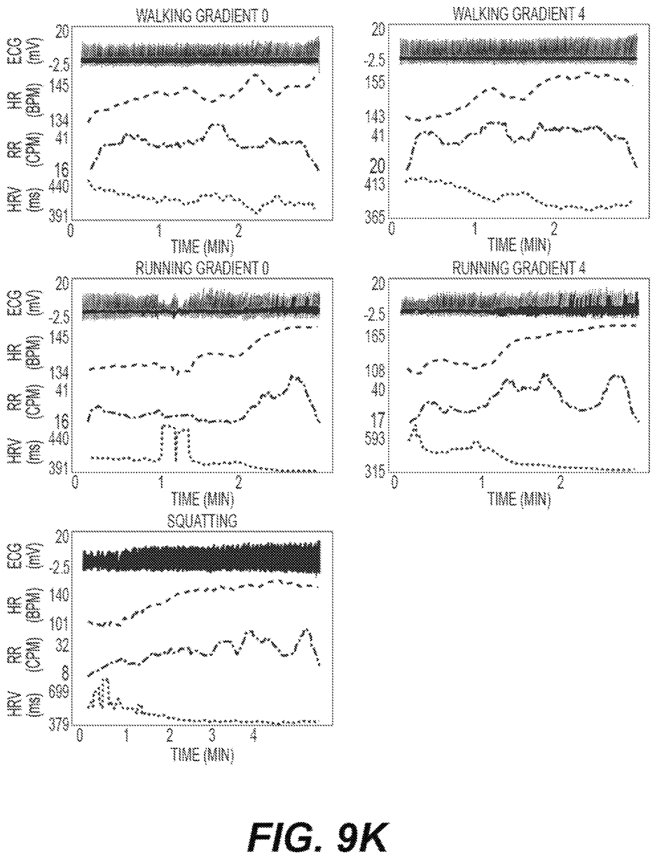

Experimental design and validation: FIGS. 9A-9C shows the signal processing and analysis of the cardiac signals to determine the metabolic cost in the study.

Details of the squatting protocol of a single session are illustrated in FIG. 9A (left) with individual squatting timing. FIG. 9A shows a timestamp and illustration of squatting and walking-running experimental protocols. The color saturation on the right side indicates elevation changes.

FIG. 9B shows representative ECG data showing 3 seconds of peak shapes and SNR during standing, squatting, walking, and running with an AFO. No significant degradation and motion artifacts in the signal qualities are observed during standing and running.

FIG. 9C shows filtered and normalized ECG data during 30 s of squatting and walking-running session (top), moving average of HR for squatting and walking-running conditions (middle), and vertical axis acceleration during activities, including standing, squatting, walking, and running (bottom).

For both squatting and walking-running, records of raw ECG data via SFB are filtered with a bandpass filter. FIG. 9D depicts an ECG filtering process for heart rate calculation. In FIG. 9D, the implementation of the algorithm is shown for a representative dataset collected by the SFB. The filtered ECG and peak data are filtered with a dynamic threshold line to eliminate incorrect ECG R peaks [26]. HR was graphed with finalized ECG peaks and then averaged to show a smooth curve. ECG signal quality is a major factor in determining the feasibility of the SFB. In response, SFB provided an overall SNR higher than 25 dB and clear distinguishable ECG R peaks for all activities, including standing, squatting, walking, and running (FIG. 9B).

FIG. 9C show the total captures of a single squatting session and walking-running session. In FIG. 9C, a 30-second capture (top) is shown with normalized ECG peaks during squatting and running, gives clear HR, and provides respiration rate (RR) for other potential calculations. Smoothed HR graphs (middle) showed an increase in HR during squatting and walking-running, where running shows the highest average HR, followed by squatting and walking.

FIGS. 9E-9G depict ECG peak shape representation for 30 seconds and average HR outcomes during the squat experiment from subject 2, subject 3, and subject 4, respectively. In FIGS. 9E-9G, more 30 s ECG peak data, and average HR outcomes during squatting from other subjects are shown to illustrate device compatibility from subject to subject. Before calculating the HRV-RMSSD, the ECG quality was compared against one of the commercially available ECG straps (Polar H10, Polar Electro).

FIGS. 9H-9J depict HR comparison with commercial HR device. FIG. 9H shows an HR graph during a single squatting session with both SFB and commercial devices. FIG. 9I shows a linear correlation between SFB-driven HR and commercial device-driven HR. FIG. 9J shows the mean and standard deviation of the HR difference between two devices. The red graph portrays the HR curve from SFB and black dots from the commercial ECG strap (FIGS. 9H-9J). At the beginning of squatting, the HR from SFB and Polar H10 shows a more significant difference due to the smoothing of a sudden HR change in the algorithm but soon converges as HR increases and reaches a steady state. The comparison results R²=0.961 for n=447 with a p-value of 0.002, and 90% of HR difference data are scattered in one standard deviation range between −2.27 and 1.72 bpm.

FIG. 9K depicts a representative subject's data of ECG, HR, RR, and HRV during standing, squatting, walking, and running of gradients 0 and 4.

Metabolic Cost Estimation Pre-processing. For the pre-processing, The study filtered the ECG signal with a first-order Butterworth bandpass filter with cutoff frequencies of 0.5 and 60 Hz. A moving average filter was then applied by convolving the signal with a sequence of length 0.15Fs and magnitude (0.15Fs)⁻¹. An RMS envelope was employed to reject periods of high noise. To detect a QRS complex, the highest local peaks spaced at least 200 milliseconds apart were identified as fiducials. These fiducials were compared to a threshold defined in Equation 2:

$$\\ \begin{matrix}Threshold = NoiseLevel + 0.25 ( SignalLevel - NoiseLevel ) & ( Eq . 2 )\end{matrix}$$

In Equation 2, NoiseLevel and SignalLevel are estimates of the noise and signal level, respectively [27]. These parameters were dynamically updated after each fiducial is classified such that if the peak is above the threshold shown in Equation 3 or Equation 4.

$$\\ \begin{matrix}SignalLevel = 0.12 5 Peak + 0.875 SignalLevel & ( Eq . 3 )\end{matrix}$$

When no QRS is detected within 166% of the average R peak interval for the previous nine beats, the maximal fiducial within this period was added as a QRS complex if it exceeded half the threshold value. Fiducials within 360 milliseconds of a QRS that exceed the threshold were rejected if the slope is less than half the average of the previous 9 QRS complexes, which can indicate that it is likely a T wave with abnormally high amplitude. The HRV-RMSSD was used for a short-term interval of 30 seconds [28]. The average RMSSD calculated from the 30-second intervals from the last 2 minutes of each squatting condition was compared with the steady-state metabolic cost. A single value was calculated to compare HRV-RMSSD with estimated metabolic cost to represent energy expenditure. Because metabolic cost can be the real-time varying quantity and estimated metabolic cost can increase for the first half of squatting then convergences into a single value, the converged filtered metabolic cost value in the last 2 minutes of the squatting session was used to compare with normalized HRV-RMSSD. For the cleaning and calculating HRV-RMSSD values, the neurokit2 [29] library was used for python after ECG filtering. The representative subject's ECG, HR, RR, and HRV-RMSSD data from the single session are shown in FIG. 9K, including standing, squatting, walking, and running, with a gradient of 0-4.

HRV analysis and motion classification via machine learning: FIGS. 10A-10E show data classification results for machine learning and estimation of metabolic costs and physical effort.

FIG. 10A shows the performance of the trained machine learning model and loss curve for CNN ML motion classification, showing the training accuracy curve.

FIG. 10B shows a confusion matrix showing data from the IMU sensor classifying six different motions (running, elevated running, walking, elevated walking, standing, and squatting) with an overall accuracy of 88%. FIG. 10C shows a flow chart representing a spatial CNN model with five layers of convolutions with filters of decreasing the dimension size and two layers of average pooling. FIG. 10D shows the metabolic rate from oxygen intake and carbon dioxide exhale measured with the calorimetric respiratory mask. Raw data are filtered with a bandpass filter, and steady-state values are used to compare with normalized HRVRMSSD. FIG. 10E shows normalized physical effort (PE) compared with normalized HRV-RMSSD, where PE represents the quantitative description of hardness for each trial by subjects measured using the Borg perceived exertion scale (6-20). The measured Pearson R correlation is −0.689 (p-value: 6.6e-6).

The designed model shows 91% test accuracy and 97% training accuracy (training and loss curve in FIG. 10A).

In addition, the classification had an overall accuracy of 88% (FIG. 10B), with the lowest accuracy of 74% from running and 82% from elevated walking. Standing and squatting both present accuracy of 99%. FIG. 10 C shows the details of the convolution process. The high accuracy of classification between walking, running, standing, and squatting shows that the model can successfully identify the dynamically distinct conditions. The reduction in the gradient condition's accuracy showed that the model is not confident in differentiating between gradients. This can be due to the three-axis accelerometer. In other embodiments, a gyroscope can be used in a combination of an accelerometer and a Madgwick filter to estimate the Euler angle and use the information as part of motion classification. The raw metabolic cost was also measured and filtered with a fourth-order bandpass filter [27], [31], as shown in FIG. 10D. Each subject's metabolic cost for every squatting condition is compared against the HRV values. Further, perceived effort (PE), a clinical term to determine the hardness of action described with numbers between 6 and 20, was used for each subject to record the user's perceived physical effort feedback (Borg rate perceived effort scale) [32]. The user's perceived efforts were subjective and varied substantially from subject to subject. The quantified perceived effort by each subject and its relationship to HRV-RMSSD indicated the overall participants' ability to recognize the step loading from the AFO. The Pearson R correlation between normalized metabolic cost and normalized PE had R=−0.689 with a p-value of 6.6×10⁻⁶ and an acceptable negative correlation (FIG. 10E). TABLE 2 shows a comparison the instant exosuit study to estimate metabolic costs and physical effort. The summary shows the limitation of current studies using respirometer and HRV studies due to their form factor and application, and there yet has not been an attempt to relate HRV-RMSSD and metabolic cost.

TABLE 2

| | | | | Metabolic | | |
|---|---|---|---|---|---|---|
| | | | | Cost to | | |
| | Metabolic | HRV- | | HRV- | | |
| | Cost | RMSSD | Biosensor | RMSSD | | |
| Reference | Estimation | Calculation | Form Factor | Correlation | Activity | Application |
| Instant | ✓ | ✓ | soft, low-profile | −0.758 | walking, | activity |
| study | | | biopatch | | running, | exoskeleton |
| | | | | | squatting | |
| [38] | — | ✓ | lead II wired | n/a | walking | robot- |
| | | | ECG | | | assisted gait |
| | | | | | | training |
| [39] | — | ✓ | lead II wired | n/a | passive | passive |
| | | | ECG | | leg | lower limb |
| | | | | | movement | |
| [40] | — | ✓ | portable lead II | n/a | walking, | robotic |
| | | | wired ECG | | mild | locomotor |
| | | | | | training | |
| [5] | ✓ | — | — | n/a | walking | walking |
| | | | | | | exoskeleton |
| [41] | ✓ | — | — | n/a | walking | lower limb |
| | | | | | | exoskeleton |
| [42] | ✓ | — | — | n/a | walking, | hip flexion |
| | | | | | running | assistant |
| [43] | ✓ | — | — | n/a | assisted | oscillator |
| | | | | | walking | |
| [12] | ✓ | — | — | n/a | walking | hip |
| | | | | | | assistance |
| | | | | | | exoskeleton |
| [44] | ✓ | — | — | n/a | loaded | walking |
| | | | | | walking | exoskeleton |

### Discussion

With all the efforts to estimate metabolic cost through mask indirect calorimetry, studies that use commercially available devices require the rigid mask to be well-fitted for accurate measurement in addition to an antenna communication system on the user's back. These bulky systems directly counter the purpose of the development of wearable robots supporting workers' repetitive motion where they take a long setup and estimation time, are confined to lab settings, and are cumbersome to wear with a large form factor and heavyweight. In addition, mask-based indirect calorimetry is slow to determine a physiological response [16], [17] while taking a longer time (>3 min) than electrocardiogram (ECG) processing [18]. It makes the wearable robot challenging to compute user response rapidly in a potential field-deployable system. A different approach has been made to optimize human parameters by estimating cognitive effort with HRV-RMSSD driven by ECG. An attempt to measure work intensity with an HR strap is reported [19]. However, commercial HR detection devices are intrinsically uncomfortable to wear and prevent the user's natural motion range, introducing more motion artifacts due to their rigid form and lack of conformal contact with the skin [20]. Further, the inherent manufacturing complexity of epidermal electronics, specifically electrode fabrication [21], [22], has been a critical factor in preventing mass manufacture. Thus, an easily customizable low-cost electrode fabrication has been introduced utilizing a high-resolution micromachining tool.

In contrast, the exemplary study employed a portable soft flexible biopatch with ankle-foot-orthosis (AFO) as an alternative to the needs of metabolic cost estimation, discovering the relationship between metabolic rate from indirect respiratory calorimetry and HRV-RMSSD from the biopatch measuring high-quality HR, ECG, and motion. The system can replace the indirect calorimetry with a low profile, small form factor, and lightweight soft flexible biopatch (SFB), providing better comfort and movement advantages. The feasibility of SFB was validated by comparing it with commercial mask indirect calorimetry while wearing AFO during a set of squatting, running, and walking experiments. The ECG quality was validated with a signal-to-noise ratio (SNR) and HR comparison with a commercial HR chest strap. Further, the physical durability and stability of SFB were computationally calculated and experimentally proved. The fabrication process in the study was demonstrated to be superior in manufacturability and ease of prototyping in contrast to conventional cleanroom processed flexible devices.

### Example Trained Machine Learning Model

Machine Learning. In addition to the machine learning model described above, the analysis system can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Example Computing System

It should be appreciated that the logical operations described above and in the appendix can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

### CONCLUSION

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

The following patents, applications and publications as listed below and throughout this document are hereby incorporated by reference in their entirety herein.

### REFERENCE LIST

[1] Bunn, P. D. S., Silva, G. D. P. & Silva, E. B. D. Performance in the Deep Squat Test and musculoskeletal injuries: a systematic review. Fisioter. mov. 31, e003126 (2018).
[2] Yan, Z. et al. Development and testing of a wearable passive lower-limb support exoskeleton to support industrial workers. Biocybern. Biomed. Eng. 41, 221-238 (2021).
[3] Sado, F., Yap, H. J., Ghazilla, R. A. R. & Ahmad, N. Design and control of a wearable lower-body exoskeleton for squatting and walking assistance in manual handling works. Mechatronics 63, 102272 (2019).
[4] Kantharaju, P. et al. Reducing squat physical effort using personalized assistance from an ankle exoskeleton. IEEE Trans. Neural Syst. Rehab. Eng. 30, 1786-1795 (2022).
[5] Shepertycky, M., Burton, S., Dickson, A., Liu, Y.-F. & Li, Q. Removing energy with an exoskeleton reduces the metabolic cost of walking. Science 372, 957-960 (2021).
[6] Wang, Z. et al. A Semi-active exoskeleton based on EMGs reduces muscle fatigue when squatting. Front. Neurorobot. https://doi.org/10.3389/fnbot.2021.625479 (2021).
[7] Wei, W., Zha, S., Xia, Y., Gu, J. & Lin, X. A hip active assisted exoskeleton that assists the semi-squat lifting. Appl. Sci. 10, 2424 (2020).
[8] Cho, Y. K., Kim, K., Ma, S. & Ueda, J. A robotic wearable exoskeleton for con-struction worker's safety and health. ASCE Constr. Res. Congr. 19-28 (2018).
[9] Cantù, E. et al. Printed multi-EMG electrodes on the 3D surface of an orthosis for rehabilitation: a feasibility study. IEEE Sens. J. 21, 14407-14417 (2021).
[10]. Sutandi, A. C. & Rahman, S. F. Robotic exosuit to improve walking and gait rehabilitation for stroke survivors: a review. AIP Conf. Proc. 2344, 050014 (2021).
[11] Jacobson, M. et al. Foot contact forces can be used to personalize a wearable robot during human walking. Sci. Rep. 12, 1-12 (2022).
[12] Ding, Y., Kim, M., Kuindersma, S. & Walsh, C. J. Human-in-the-loop optimization of hip assistance with a soft exosuit during walking. Sci. Robot. 3, eaar5438 (2018).
[13] Zhang, J. et al. Human-in-the-loop optimization of exoskeleton assistance during walking. Science 356, 1280-1284 (2017).
[14] Kim, M. et al. Human-in-the-loop Bayesian optimization of wearable device parameters. PLOS One 12, e0184054 (2017).
[15] Ross, R., ALDuhishy, A. & González-Haro, C. Validation of the cosmed K4b2 por-table metabolic system during running outdoors. J. Strength Cond. Res. 34, 124-133 (2020).
[16] Badesa, F. J. et al. Physiological responses during hybrid BNCI control of an upper-limb exoskeleton. Sensors 19, 4931 (2019).
[17] Young, A. J. & Ferris, D. P. State of the art and future directions for lower limb robotic exoskeletons. IEEE Trans. Neural Syst. Rehab. Eng. 25, 171-182 (2016).
[18] Ingraham, K. A., Rouse, E. J. & Remy, C. D. Accelerating the estimation of meta-bolic cost using signal derivatives: implications for optimization and evaluation of wearable robots. IEEE Robot. Autom. Mag. 27, 32-42 (2020).
[19] Villani, V., Righi, M., Sabattini, L. & Secchi, C. Wearable devices for the assessment of cognitive effort for human-robot interaction. IEEE Sens. J. 20, 13047-13056 (2020).
[20] Rodeheaver, N. et al. Strain-isolating materials and interfacial physics for soft wearable bioelectronics and wireless, motion artifact-controlled health mon-itoring. Adv. Funct. Mater. 31, 2104070 (2021).
[21] Wang, W. et al. Strain-insensitive intrinsically stretchable transistors and circuits. Nat. Electron. 4, 143-150 (2021).
[22] Song, J.-K. et al. Stretchable colour-sensitive quantum dot nanocomposites for shape-tunable multiplexed phototransistor arrays. Nat. Nanotechnol. 17, 849-856 (2022).
[23] Kim, D.-H. et al. Epidermal electronics. Science 333, 838-843 (2011).
[24] Kim, Y. S. et al. Soft wireless bioelectronics designed for real-time, continuous health monitoring of farmworkers. Adv. Healthc. Mater. 11, 2200170 (2022).
[25] Brockway, J. Derivation of formulae used to calculate energy expenditure in man. Hum. Nutr. Clin. Nutr. 41, 463-471 (1987).
[26] Kalidas, V. & Tamil, L. Real-time QRS detector using stationary wavelet transform for automated ECG analysis. IEEE Int. Conf. Bioinformatics Bioeng. (BIBE). 457-461 (2017).
[27] Pan, J. & Tompkins, W. J. A real-time QRS detection algorithm. IEEE Trans. Biomed. Eng. BME-32, 230-236 (1985).
[28] Castaldo, R., Montesinos, L., Melillo, P., James, C. & Pecchia, L. Ultra-short term HRV features as surrogates of short term HRV: a case study on mental stress detection in real life. BMC Med. Inf. Decis. Mak. 19, 12 (2019).
[29] Makowski, D. et al. NeuroKit2: A Python toolbox for neurophysiological signal processing. Behav. Res. Methods 53, 1689-1696 (2021).
[30] Madgwick, S. O., Harrison, A. J. & Vaidyanathan, R. Estimation of IMU and MARG orientation using a gradient descent algorithm. IEEE Int. Conf. Rehab. Robot. 1-7 (2011).
[31] Hamilton, P. Open source ECG analysis. IEEE Comput. Cardiol. 101-104 (2002).
[32] Gunnar, A. B. Psychophysical bases of perceived exertion. Med. Sci. Sports Exerc. 14, 377-381 (1982).
[33] Mukaka, M. M. A guide to appropriate use of correlation coefficient in medical research. Malawi Med. J. 24, 69-71 (2012).
[34] Rahimi, R. Effect of different rest intervals on the exercise volume completed during squat bouts. J. Sports Sci. Med. 4, 361-366 (2005).
[35] Escamilla, R. F., Fleisig, G. S., Lowry, T. M., Barrentine, S. W. & Andrews, J. R. A three-dimensional biomechanical analysis of the squat during varying stance widths. Med. Sci. Sports Exerc. 33, 984-998 (2001).
[36] Kushner, A. M. et al. The back squat part 2: targeted training techniques to correct functional deficits and technical factors that limit performance. Strength Cond. J. 37, 13-60 (2015).
[37] Paszke, A. et al. Pytorch: An imperative style, high-performance deep learning library. Adv. Neural Inf. Process. Syst. 32, 4399 (2019).
[38] Jelinek, H. F. et al. Cortical response to psycho-physiological changes in auto-adaptive robot assisted gait training. IEEE Eng. Med. Biol. Soc. 7409-7412 (2011).
[39] Shi, P., Li, A., Wu, L. & Yu, H. The effect of passive lower limb training on heart rate asymmetry. Physiol. Meas. 43, 015003 (2022).
[40] Evans, R. W. et al. Robotic locomotor training leads to cardiovascular changes in individuals with incomplete spinal cord injury over a 24-week rehabilitation period: A randomized controlled pilot study. Arch. Phys. Med. Rehabil. 102, 1447-1456 (2021).
[41] Quinlivan, B. T. et al. Assistance magnitude versus metabolic cost reductions for a tethered multiarticular soft exosuit. Sci. Robot. 2, eaah4416 (2017).
[42] Chen, L. et al. A portable waist-loaded soft exosuit for hip flexion assistance with running. Micromachines 13, 157 (2022).
[43] Schroeder, R. T., Croft, J. L. & Bertram, J. E. A. Evaluating the energetics of entrainment in a human-machine coupled oscillator system. Sci. Rep. 11, 15804 (2021).
[44] Panizzolo, F. A. et al. A biologically-inspired multi-joint soft exosuit that can reduce the energy cost of loaded walking. J. Neuroeng. Rehab. 13, 43 (2016).

What is claimed is:

1. A system comprising:
a processor; and
a memory having instructions stored thereon, wherein the instructions when executed by the processor causes the processor to:
receive a cardiac signal data set acquired by a electrode measurement device placed on a subject while the subject was performing an activity;
determine heart rate variability signal or value using the cardiac signal data set; and
determine, via a trained machine learning model, a value for metabolic cost for the activity, wherein the trained machine learning model was trained using metabolic cost data and heart rate variability data,
wherein the determined value for metabolic cost is made accessible to be used to an evaluation of the activity.

2. The system of claim 1, wherein the cardiac signal data set is acquired from ECG equipment or a wearable sensor device.

3. The system of claim 2, wherein the wearable sensor device includes an array of stretchable electrodes, the array comprising a first side and a second side, a plurality of interconnectors joining the electrodes of the array of stretchable electrodes, and an adhesive patch on the first side of the array of stretchable electrodes, the adhesive patch configured to hold the second side of the array of stretchable electrodes to a surface of a patient's skin.

4. The system of claim 1, wherein the instructions, when executed by the processor, causes the processor to determine via a second trained machine learning model a motion classification output from an acquired motion signal data set acquired via the electrode measurement device.

5. The system of claim 1, wherein the instructions, when executed by the processor, causes the processor to output the value of the metabolic cost in a graphical user interface or a report to be used for an evaluation of the activity.

6. The system of claim 1, wherein the system is configured as a smartwatch or smartphone.

7. The system of claim 1, wherein the system is implemented in cloud infrastructure.

8. A method comprising:
receiving a cardiac signal data set acquired by an electrode measurement device placed on a subject while the subject was performing an activity;
determining heart rate variability signal or value using the cardiac signal data set; and
determining, via a trained machine learning model, a value for metabolic cost for the activity, wherein the trained machine learning model was trained using metabolic cost data and heart rate variability data,
wherein the determined value for metabolic cost is made accessible to be used to an evaluation of the activity.

9. The method of claim 8, wherein the cardiac signal data set is acquired from ECG equipment or a wearable sensor device.

10. The method of claim 9, wherein the wearable sensor device includes an array of stretchable electrodes, the array comprising a first side and a second side, a plurality of interconnectors joining the electrodes of the array of stretchable electrodes, and an adhesive patch on the first side of the array of stretchable electrodes, the adhesive patch configured to hold the second side of the array of stretchable electrodes to a surface of a patient's skin.

11. The method of claim 8 further comprising:
determining via a second trained machine learning model a motion classification output from an acquired motion signal data set acquired via the electrode measurement device.

12. The method of claim 8 further comprising:
outputting the value of the metabolic cost in a graphical user interface or a report to be used for an evaluation of the activity.

13. The method of claim 8, wherein the system is configured as a smartwatch or smartphone.

14. The method of claim 8, wherein the system is implemented in cloud infrastructure.

15. The method of claim 8 further comprising:
acquiring the cardiac sensor at the electrode measurement device placed on the subject while the subject was performing the activity; and
transmitting the acquired cardiac sensor to an analysis system on a remote computing device to perform the determining the value for metabolic cost for the activity.

16. The method of claim 8 further comprising:
acquiring the cardiac sensor at the electrode measurement device placed on the subject while the subject was performing the activity; and
transmitting the acquired cardiac sensor to a computing device that then transmits the acquired cardiac sensor to an analysis system on a remote computing device to perform the determining the value for metabolic cost for the activity, wherein the determined value for the metabolic cost is transmitted from the remote computing device to the computing device.

17. The method of claim 8, wherein the value of the metabolic cost is employed as a measure of physical exertion.

18. A non-transitory computer-readable medium having instructions stored thereon, wherein the instructions when executed by a processor causes the processor to:
receive a cardiac signal data set acquired by a electrode measurement device placed on a subject while the subject was performing an activity;
determine heart rate variability signal or value using the cardiac signal data set; and
determine, via a trained machine learning model, a value for metabolic cost for the activity, wherein the trained machine learning model was trained using metabolic cost data and heart rate variability data,
wherein the determined value for metabolic cost is made accessible to be used to an evaluation of the activity.

19. The non-transitory computer-readable medium of claim 18, wherein the cardiac signal data set is acquired from ECG equipment or a wearable sensor device.

20. The non-transitory computer-readable medium of claim 18, wherein the instructions, when executed by the processor, causes the processor to output the value of the metabolic cost in a graphical user interface or a report to be used for an evaluation of the activity.
